(19) United States
(12) Patent Application Publication
(10) Pub. No.: US 2005/0079088 A1
(43) Pub. Date: Apr. 14, 2005

(54) Medical implants, prostheses, prosthesis parts, medical instruments, devices and auxiliary contrivances made of a halogenide-modified magnesium substance

(76) Inventors: Carl-Joachim Wirth, Isernhagen (DE); Henning Windhagen, Hannover (DE); Frank Witte, Hannover (DE); Friedrich-Wilhelm Bach, Isernhagen (DE); Volker Kaese, Hannover (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/499,992

(22) PCT Filed: Dec. 13, 2002
(86) PCT No.: PCT/DE02/04568

(30) Foreign Application Priority Data
Dec. 24, 2001 (DE) 101 63 106.5

(51) Int. Cl. C22C 23/02; A61F 2/28; C22C 23/06

(52) U.S. Cl. 420/402; 623/23.53; 420/405

(57) ABSTRACT

The use of a magnesium substance, whose corrosiveness is altered as a result of modification with halogenides, enables medical implants, prostheses or prosthesis parts and medical instruments, devices and auxiliary contrivances, especially surgical instruments or tools, for use in and on the human or animal body to be produced, whereby the degree of corrosion-resistance thereof can be adjusted to full corrosion-resistance.

The invention relates to the use of a special magnesium substance, whose corrosiveness has been altered, for producing medical implants for use in or on the human or animal body, prosthesis parts, prostheses, medical instruments, devices and auxiliaries, and also to these implants, prostheses, instruments, devices and auxiliaries themselves.

The invention relates in the wider sense to absorbable implants used for releasing medicaments or as securing elements in hard and soft tissue.

Absorbable implants have to date been represented by polymers. However, these have two serious disadvantages. On the one hand, plasticizers harmful to the body are released during the release, and, on the other hand, the mechanical properties of the polymers are unsatisfactory.

Since the beginning of the 20th century it has been known that implants made of magnesium and its alloys afford advantages because of the fact that they are easily absorbable and biocompatible. The absorbability in the body is based on the corrosion of magnesium in saline immersions. Its essential character for the body's functions and the elimination of excess doses via the urinary tract qualify magnesium as a basic implant substance with a high level of physical and chemical biocompatibility. The average distribution in the body mass is 470 mg/kg, the recommended daily dose is 200 to 300 mg/d MgSO₄. Magnesium also has an antiarrhythmic effect and lowers blood pressure and sensitivity to pain. The maximum dose for short-term infusion in a human weighing 75 kg is 57.6 mg pure magnesium. Blood plasma contains, for example, 107 mMol/l, and gastric juice contains 160 mval/l of magnesium chloride ions, so that magnesium will corrode in these saline immersions and, as a result, permit metallic and mechanically loadable but biodegradable temporary implants.

The developments in the first Mg period before World War Two (Verbrugge 1933, McBride 1938, Lambotte 1932) were unable to provide any alloys which corrode sufficiently slowly and thus develop only small amounts of hydrogen biocompatability. The developments in the second Mg period during the Cold War (Stroganov, DE-OS 1 953 241) provided alloys with greater corrosion resistance whose cadmium additive present in principle was intended to accelerate bone fusion, but whose toxicity meant that it could not be implanted in western industrialized nations under anthropological aspects. However, these alloys were strongly inhibited in respect of their speed of corrosion and developed hydrogen to an extent which was biocompatible.

Other more recent magnesium alloys for absorbable implants contain rare earth metals, preferably in addition to lithium. With these alloys, the absorption of the implant is already considerably delayed, but with these materials, too, there is still appreciable development of hydrogen and gas pockets in the tissue. For many applications, the rate of corrosion of the alloys containing rare earth metals is still too high, since the stability losses associated with absorption occur too early in the healing or tissue formation process.

There is therefore a need for absorbable magnesium substances for medical implants whose absorption is delayed compared to the previously known substances. It would be particularly advantageous if the absorption could be adapted to the site of application and the intended use.

Compared to steel and titanium implants, magnesium implants also have considerable advantages in respect of their mechanical properties, in particular their stability. In the case of direct connection of the implant to human bone, the aim is for the stability of the implant substance to be adapted to the strength of the bone. Magnesium alloys per se have elasticity moduli similar to bone, so that it is possible to come closer to the desired isoelasticity between implant and bone.

There is therefore also a great need for completely corrosion-resistant magnesium substances which could be used for permanent implants, prostheses, and medical instruments and devices coming into contact with body fluid and/or body tissues.

The object of the invention is therefore to find magnesium substances for medical implants, prostheses and/or prosthesis parts and medical instruments and devices, in particular surgical instruments and tools, these substances having a corrosion resistance which can be set to full corrosion resistance.

This object is achieved by the use of a magnesium substance, whose corrosiveness has been altered by modification with halides, for producing medical implants, prosthesis parts, prostheses and medical instruments, devices and auxiliaries.

The previously known magnesium substances are absorbable rapidly or in the medium term. By modification with halides, the corrosion resistance is increased, specifically more so the greater the concentration of halide ions in the substance as a whole or in the workpiece surface. The corrosion resistance can be adapted so that the absorption desired for the intended use can be set. Starting from a fluoride ion proportion of ca. 10 to 12 at. %, the substance or the area influenced by this concentration can be regarded as completely corrosion-resistant over the lifetime of the implant, prosthesis or medical instrument or device. In this way, the advantageous mechanical properties of magnesium substances can be utilized also for permanent implants, prostheses, prosthesis parts, instruments, devices, etc.

For medical devices, in particular surgical instruments and devices such as operating equipment, tools and devices, the halide-modified magnesium substance used here also has considerable advantages because it is not magnetic, so that instruments and devices, etc., made from it can be used for operations performed in the range of activity of an MRI unit. The substance is also readily visible in radiography and is therefore also suitable for use in computed tomography.

According to this invention, the modification of the magnesium substances known as such is effected by alloying of halogen with the aid of halogen compound salts, namely halides. Incorporation of halides and thus of halide ions into the substance can be performed in the course of normal alloying processes.

Fluorides are preferably used. In addition to fluorides, it is possible in particular to use the chlorides similar to fluorides.

The fluorides used are preferably those metal fluorides and complex metal fluorides which are thermodynamically less stable than MgF₂, CaF₂ and LiF, so that MgF₂, CaF₂ and LiF can form in the course of the alloy formation. AlF₃, KBF₄ and Na₃AlF₆ are preferably used. The concentration of the fluorides in the magnesium substance is preferably set at 1 to 15 at. % F, more preferably 1.5 to 2.5 at. %.

The salts can be introduced by various routes into the magnesium substance, for example by gas alloying, melt alloying, mechanical alloying, centrigugal casting, reaction milling, and diffusion alloying, which are cited here are representative examples for other techniques. As is known for magnesium substances, the halogen-modified substance can be additionally treated, for example by thermomechanical processes, by sequential extrusion, homogenization and age-hardening. The material can be worked by cutting or shaping, e.g. by rolling, turning, forging or punching.

In diffusion alloying, semifinished products are treated for a specified time at elevated pressure and elevated temperature. Semifinished products made of a magnesium substance, i.e. of pure magnesium or a magnesium-based alloy, are preferably embedded in a halide, preferably aluminum fluoride (AlF₃), and diffusion-alloyed at temperatures of up to 850° C., preferably 420° C, e.g. for 24 hours. The diffusion alloying processes include the powder packing technique. As it was possible to demonstrate with the aid of immersion tests in aggressive synthetic sea water, diffusion alloying produces corrosion-stable coatings which provide protection in the pH range between 3 and 14.

According to the invention, melt alloying is performed with an addition of halides to the metal melt, preferably with addition of 1 to 15 at. % F, more preferably with 1.5 to 2.5 at. % F. In a preferred embodiment, AlF₃ is used.

In a development of the invention, the halide-modified magnesium substance can be applied to the metal surface of a workpiece, in particular by vapor deposition or by metal-spraying or sintering techniques onto prefabricated implants, prostheses and medical instruments and devices. CVD or PVD processes, thermal spraying in arc or plasma and co-extrusion are suitable, inter alia.

In the context of the present invention, the magnesium substance used is pure magnesium or a magnesium alloy which contains proportions of lithium and/or calcium and/or aluminum and/or rare earth metals.

Both magnesium and also calcium and lithium form stable halides which, on the surface of a workpiece, can form a coating providing protection against corrosion. For example, when the presently preferred AlF₃ is used as process salt, the thermodynamically more stable salts MgF₂, CaF₂ and LiF form during alloying. The magnesium substance preferably contains lithium in a proportion of 0 to 7 wt. %, aluminum in a proportion of 0 to 16 wt. %, calcium in a proportion of 0 to 5 wt. % and rare earth metals, preferably cerium and/or neodymium and/or praseodymium, in a proportion of 0 to 8 wt. %, and yttrium in a proportion of 0 to 7 wt. %.

Basic substances that can be modified by halogen are, in particular, LAE 442 (MgLi4Al4SE2 wt. %), MgY4SE3Li2.4 wt. %, MgLil2 at. %, MgLi40 at. %, MgCa30 at. %, AZ31 or AZ91.

The magnesium and magnesium/lithium alloys containing rare earth metals are themselves already corrosion-inhibited by the influence of the rare earth metals and can be made even more corrosion-resistant by additional alloying with halides, in particular fluorides. As such, faster-corroding magnesium base materials require a higher proportion of admixed fluoride to achieve a comparable effect. By suitable choice of the alloy partners, it is possible to obtain materials which are in practice resistant to corrosion.

The invention includes medical implants, in particular securing elements for bone, for example screws, plates or nails, anchors, pins, pacers, cages, buttons, hoops, surgical suture material, such as threads or wires, films and meshes (inter alia for wound or fracture treatment), wound clips, suture clips and intestine clips, vessel clips, abrasive particles for water-jet cutting, prostheses in the area of hard and soft tissue, associated prosthesis parts, and medical instruments and devices, in particular operating equipment, tools and devices which consist at least in part of the above-described halide-modified magnesium substances, or which are halide-modified at least on parts of their surface. Modification with up to 15 at. % F is preferred.

The fluoride quantities used are not critical for the metabolism because of the slow release; in the practically corrosion-stable alloys with a higher fluoride proportion, fluoride is released only negligibly over lengthy periods of time.

An example of a halide-modified magnesium substance to be used according to the invention is given below.

### EXAMPLE

Basic material LAE442 (MgLi4Al4SE2 wt. %), melt-alloyed in a crucible with 2 at. % AlF₃.

The fluoride-modified alloy has a 10-fold improved corrosion resistance in aggressive electrolytes (synthetic sea water as test medium, comparable results with 5% strength NaCl solution) and satisfactory mechanical parameters even in the cast state:
R_{p0.2}=80 MPa
Rₘ=180 MPa
A₅=8%

1. Use of a magnesium substance, whose corrosiveness is altered by modification with halides, for producing medical implants, prosthesis parts, prostheses, medical instruments, devices and auxiliaries for use in or on the human or animal body.

2. The use as claimed in claim 1, characterized in that fluorides or chlorides, preferably fluorides, are used for the modification.

3. The use as claimed in claim 2, characterized in that KBF₄, Na₃AIF₆ or AIF₃ are used as fluorides.

4. The use as claimed in claim 3, characterized in that the concentration of the fluorides in the magnesium substance is set at 1 to 15 at. % F, preferably 1.5 to 2.5 at. % F.

5. The use as claimed in claim 1, characterized in that the modification with halides is performed by diffusion alloying or melt alloying.

6. The use as claimed in claim 1, characterized in that the halide-modified magnesium substance is applied to the metal surface of a workpiece, in particular by vapor deposition or by metal-spraying or sintering techniques onto prefabricated implants, prostheses and medical devices.

7. The use as claimed in claim 1, characterized in that the magnesium substance is pure magnesium or a magnesium alloy which contains proportions of lithium and/or calcium and/or aluminum and/or rare earth metals.

8. The use as claimed in claim 7, characterized in that the magnesium substance contains lithium in a proportion of 0 to 7 wt. %, aluminum in a proportion of 0 to 16 wt. %, calcium in a proportion of 0 to 5 wt. %, and rare earth metals, preferably cerium and/or neodymium and/or praseodymium, in a proportion of 0 to 8 wt. %, and yttrium in a proportion of 0 to 7 wt. %, at least one alloy component with at least 0.1 at. % being included.

9. The use as claimed in claim 1, characterized in that the fluoride-modified magnesium base substance is LAE 442 (MgLi4Al4SE2) wt. %), MgY4SE3Li2.4 wt. %, MgLil2 at . %, MgLi40 at. %, MgCa30 at. %, AZ31 or AZ91.

10. A medical implant which contains at least in part a halide-modified magnesium substance.

11. The medical implant as claimed in claim 10, characterized in that the magnesium substance is a halide-modified pure magnesium or a halide-modified magnesium alloy containing proportions of lithium and/or calcium and/or aluminum and/or rare earth metals.

12. The medical implant as claimed in claim 10, characterized in that the magnesium substance contains up to 15 at. %, preferably 1.5 to 2.5 at. % halide, preferably fluoride.

13. A prosthesis or prosthesis part which contains at least in part a halide-modified magnesium substance.

14. The prosthesis or prosthesis part as claimed in claim 13, characterized in that the magnesium substance is a halide-modified pure magnesium or a halide-modified magnesium alloy containing proportions of lithium and/or calcium and/or aluminum and/or rare earth metals.

15. The prosthesis or prosthesis part as claimed in claim 13, characterized in that the magnesium substance contains up to 15 at. %, preferably 1.5 to 2.5 at. % halides, preferably fluoride.

16. A medical instrument, device or auxiliary which contains at least in part a halide-modified magnesium substance.

17. The medical instrument, device or auxiliary as claimed in claim 16, characterized in that the magnesium substance is a halide-modified pure magnesium or a halide-modified magnesium alloy containing proportions of lithium and/or calcium and/or aluminum and/or rare earth metals.

18. The medical instrument, device or auxiliary as claimed in claim 16, characterized in that the magnesium substance contains up to 15 at. % halides, preferably 1.5 to 2.5 l at. % halides, preferably fluorides.
